SEQUENCE LISTING (3 sequences)

SEQ ID NO: 1 - DNA, 20 bp, synthetic construct
  source 1..20: mol_type = other DNA
gaagcgtttatgcggaagag

SEQ ID NO: 2 - DNA, 20 bp, synthetic construct
  source 1..20: mol_type = other DNA
acctgctgatctgcgactta

SEQ ID NO: 3 - DNA, 364 bp, unidentified
  source 1..364: mol_type = genomic DNA
gaagcgtttatgcggaagaggtaaagcccttcccgagtaacaaaaaaacaacagcataaataaccccgctcttacacattccagccctgaaaaagggcatcaaattaaaccacacctatggtgtatgcatttatttgcatacattcaatcaattgttatctaaggaaatacttacatatggttcgtgcaaacaaacgcaacgaggctctacgaatcgagagtgcgttgcttaacaaaatcgcaatgcttggaactgagaagacagcggaagctgtggggagtgcgttgcttaacaaaatcgcaatgcttggaactgagaagacagcggaagctgtgggcgttgataagtcgcagatcagcaggt

(19) United States
(12) Patent Application Publication
(10) Pub. No.: US 2026/0265816 A1
(43) Pub. Date: Sep. 10, 2026

(54) MULTI-DIMENSIONAL DIGITAL POLYMERASE CHAIN REACTION ANALYSIS METHOD USING PHOTOPOLYMERIZATION TECHNOLOGY

(71) Applicants: MYONGJI UNIVERSITY INDUSTRY AND ACADEMIA COOPERATION FOUNDATION, Yongin-si (KR); HONGIK UNIVERSITY INDUSTRY-ACADEMIA COOPERATION FOUNDATION, Seoul (KR)

(72) Inventors: Dohyun KIM, Seoul (KR); Abdi Mirgissa KABA, Yongin-si (KR); Bhargav Krishna PULLAGURA, Yongin-si (KR); Minsub CHUNG, Seoul (KR); Nguyen Anh Nhung TRAN, Seoul (KR); Chan Sik OH, Seoul (KR); Seunghyun SHIN, Seoul (KR)

(73) Assignees: MYONGJI UNIVERSITY INDUSTRY AND ACADEMIA COOPERATION FOUNDATION, Yongin-si (KR); HONGIK UNIVERSITY INDUSTRY-ACADEMIA COOPERATION FOUNDATION, Seoul (KR)

(21) Appl. No.: 18/869,547

(22) PCT Filed: Oct. 3, 2024
(86) PCT No.: PCT/KR2024/015035

§ 371 Date: Nov. 26, 2024

(30) Foreign Application Priority Data
Oct. 23, 2023 (KR) 10-2023-0142467

(51) Int. Cl. C12Q 1/6851; B01L 3/00; B01L 7/00; G01N 21/64

(52) U.S. Cl. CPC C12Q 1/6851; B01L 3/502707; B01L 7/52; G01N 21/6428; G01N 21/6458; B01L 2200/0647; B01L 2200/10; B01L 2200/12; B01L 2300/0822; B01L 2300/12; G01N 2021/6439

(57) ABSTRACT

Provided is a kit for preparing a digital polymerase chain reaction (PCR) solution, including a photopolymerizable monomer, a photoinitiator, and a fluorescent probe and uses thereof. According to the present disclosure, in the case of using the kit for preparing the digital PCR solution, the digital PCR solution may be partitioned into a plurality of isolators by continuously forming patterns in which solution partitions and gel walls are repeated in a light-transmitting container having a predetermined space without a microfluidic droplet partition step. Further, in the case of using the method for detecting or quantifying nucleic acids based on photopolymerization, it is possible to three-dimensionally partition the digital PCR solution and simply and rapidly acquire fluorescence data. Therefore, the method for detecting or quantifying nucleic acids based on photopolymerization according to the present disclosure may implement digital PCR with improved speed, integration, precision, sensitivity, and specificity.

### TECHNICAL FIELD

The present disclosure relates to a kit for preparing a digital PCR solution, and more particularly, to a kit for preparing a digital PCR solution that may be partitioned into a plurality of isolators by photopolymerization, and a method for detecting or quantifying nucleic acids and a device for detecting or quantifying nucleic acids as uses thereof.

### BACKGROUND ART

Digital polymerase chain reaction (PCR) (dPCR) is a third-generation PCR technology that partitions a PCR sample into thousands to hundreds of thousands of samples, amplifies 0 or one or more nucleic acid molecules present in each of the partitioned samples, and then counts the partitioned samples that show a positive reaction to measure the amount of specimen nucleic acids. The digital PCR (dPCR) has an advantage of calculating the absolute number of nucleic acids present in the PCR sample. However, in order to obtain accurate results through the digital PCR (dPCR), a process of partitioning the PCR sample into thousands to hundreds of thousands of samples is required, which has disadvantages of causing long work and detection times, expensive equipment, and a relatively narrow range of detection concentration.

The digital PCR (dPCR) method is largely classified into droplet digital PCR (ddPCR) and chamber digital PCR (cdPCR) depending on a method of partitioning a PCR sample. The ddPCR is a method of partitioning a PCR sample using droplet microfluidics, which sequentially partitions the PCR sample in flowing oil to generate reaction droplets. The ddPCR has several disadvantages, such as a long droplet generation time, non-uniformity of the generated droplets, errors in measurement signals due to combination of the generated droplets, difficulty in acquiring data due to continuous movement of droplets, and the risk of sample loss. The cdPCR is a method for partitioning a PCR sample using microchambers and microchannels manufactured using microprocessing technology, in which the PCR sample is transferred and partitioned into thousands to tens of thousands of microchambers using capillary phenomena, etc., inside a microchip. The cdPCR has several disadvantages, such as high loss of the PCR sample occurring during the partitioning process, errors in result values occurring when some of the PCR samples are not filled in the microchambers, and the partition number of the PCR sample limited to tens of thousands of samples due to the number of microchambers.

As a prior art of digital PCR (dPCR), the literature [Eleen Y. Shum et al. Anal. Chem. 2022, 94, 17868-17876] discloses a method of generating droplets using centrifugation and then counting the amplified droplets by irradiating a PCR tube with a light sheet. The prior art uses the light sheet to read the amplified signals, and has errors in the result values because the droplets are unstable and randomly distributed in a three-dimensional space.

### DISCLOSURE

### Technical Problem

The present disclosure is derived under the conventional technical background, and an object of the present disclosure is to provide a kit for preparing a digital polymerase chain reaction (PCR) solution that may be partitioned into a plurality of isolators by photopolymerization.

In addition, another object of the present disclosure is to provide a method for detecting or quantifying nucleic acids using photopolymerization technology.

In addition, yet another object of the present disclosure is to provide a device for detecting or quantifying nucleic acids using photopolymerization technology.

### Technical Solution

In order to overcome the disadvantages of conventional dPCR, the present inventors developed a method for analyzing nucleic acid samples with higher precision without using microfluidics technology. Specifically, the present inventors developed a digital PCR method based on photopolymerization technology, which overcomes the integration limit of conventional dPCR and has reduced errors, and improved dynamic range, accuracy, sensitivity, and specificity by photopolymerizing a digital PCR solution containing nucleic acids using photolithography technology to partition the digital PCR solution into a grid pattern in which solution partitions and gel walls are repeated, performing a PCR reaction on each isolator generated by partitioning the digital PCR solution, and then counting the isolators showing an amplification reaction.

In order to solve the problems, one embodiment of the present disclosure provides a kit for preparing a digital polymerase chain reaction (PCR) solution, including a photopolymerizable monomer, a photoinitiator, and a fluorescent probe.

The photopolymerizable monomer may form a gel polymer by photopolymerization. In addition, the digital PCR solution may be partitioned into a plurality of isolators by photopolymerization. The isolator consists of a gel wall formed by photopolymerization and a solution partition partitioned by the wall and capable of polymerization reaction.

In order to solve the problem, one embodiment of the present disclosure provides a method for detecting or quantifying nucleic acids based on photopolymerization including: (a) preparing a light-transmitting PCR container containing a digital PCR solution; (b) irradiating light to the light-transmitting PCR container containing the digital PCR solution to induce photopolymerization so that the digital PCR solution is partitioned into a pattern in which solution partitions and gel walls are repeated, and generate a plurality of isolators; (c) applying heat to the light-transmitting PCR container to amplify nucleic acids present in the solution partitions for each isolator after partitioning the digital PCR solution; and (d) acquiring fluorescence data from the partitioned isolators using a fluorescence imaging technique, after amplifying the nucleic acids.

The digital PCR solution may include a target nucleic acid-containing sample, a primer, a photopolymerizable monomer, a photoinitiator, a fluorescent probe, a DNA polymerase, a deoxynucleoside triphosphate (dNTP), a metal ion-providing compound, and a buffer solution.

In order to solve the problem, one embodiment of the present disclosure provides a device for detecting or quantifying nucleic acids including the kit for preparing the digital PCR

### Solution

The device for detecting or quantifying nucleic acids may optionally further include a photolithography system or a fluorescence imaging system.

### Advantageous Effects

According to the present disclosure, in the case of using the kit for preparing the digital PCR solution, the digital PCR solution may be partitioned into a plurality of isolators by continuously forming patterns in which solution partitions and gel walls are repeated in a light-transmitting container having a predetermined space without a microfluidic droplet partition step. Further, in the case of using the method for detecting or quantifying nucleic acids based on photopolymerization, it is possible to three-dimensionally partition the digital PCR solution and simply and rapidly obtain fluorescence data. Therefore, according to the present disclosure, the method for detecting or quantifying nucleic acids based on photopolymerization can implement digital PCR with improved speed, integration, precision, sensitivity, and specificity.

### DESCRIPTION OF DRAWINGS

FIG. 1 schematically illustrates a configuration of a light-sheet photolithography system.

FIG. 2 is a result acquired by performing a process for detecting and quantifying nucleic acids using 1D dPCR in an embodiment of the present disclosure. FIG. 2A is an image of a 1D dPCR microfluidic chip made of glass, FIG. 2B is a fluorescence image acquired by scanning a microfluidic channel after completing amplification of nucleic acids using a thermal cycler, FIG. 2C is an enlarged portion of the fluorescence image of FIG. 2B, and FIG. 2D illustrates a distribution of fluorescence signal intensities of each isolator partitioned in a microfluidic channel when the amplification of nucleic acids using the thermal cycler is completed.

FIG. 3 is a schematic diagram of a microfluidic chip used when performing a process for detecting and quantifying nucleic acids using 2D dPCR in an embodiment of the present disclosure, and isolators in a 2-dimensional grid pattern formed in a microfluidic chamber of the microfluidic chip.

FIG. 4 is a result acquired by performing a process for detecting and quantifying nucleic acids using 2D dPCR in an embodiment of the present disclosure.

FIG. 5 is a schematic diagram illustrating a method for detecting and quantifying nucleic acids using a photopolymerization-based 3D dPCR proposed in the present disclosure step by step.

FIG. 6 illustrates a process for generating a plurality of isolators by 3-dimensionally partitioning a polymerase chain reaction (PCR) solution contained in a predetermined container using a light-sheet photolithography system.

FIG. 7 illustrates a process for acquiring fluorescence data from isolators partitioned into a 3-dimensional grid pattern using a light-sheet fluorescence microscopy (LSFM) system.

### MODES OF THE INVENTION

Hereinafter, the present disclosure will be described in detail.

As used in the present disclosure, the term ‘1-dimensional digital polymerase chain reaction (1D dPCR)’ or ‘1-dimensionally partitioning’ or ‘partitioning into a 1-dimensional pattern’ includes a process of partitioning a digital polymerase chain reaction (PCR) solution into a plurality of isolators by forming a pattern in which solution partitions and gel walls surrounding or partitioning the solution partitions are repeated in one direction (for example, X-axial direction) in the digital PCR solution.

As used in the present disclosure, the term ‘2-dimensional digital polymerase chain reaction (2D dPCR)’ or ‘2-dimensional partitioning’ or ‘partitioning into a 2-dimensional pattern’ includes a process of partitioning a digital PCR solution into a plurality of isolators by forming a pattern in which solution partitions and gel walls surrounding the solution partitions are repeated in two directions (for example, X-axial direction and Y-axial direction) in the digital PCR solution.

As used in the present disclosure, the term ‘3-dimensional digital polymerase chain reaction (3D dPCR)’ or ‘3-dimensional partitioning’ or ‘partitioning into a 3-dimensional pattern’ includes a process of partitioning a digital PCR solution into a plurality of isolators by forming a pattern in which solution partitions and gel walls surrounding the solution partitions are repeated in three directions (for example, X-axial direction, Y-axial direction, and Z-axial direction) in the digital PCR solution.

One aspect of the present disclosure relates to a kit for preparing a digital polymerase chain reaction (PCR) solution.

The kit for preparing the digital PCR solution according to an embodiment of the present disclosure includes a photopolymerizable monomer, a photoinitiator, and a fluorescent probe. The digital PCR solution may be partitioned into a plurality of isolators by photopolymerization. The isolator consists of a gel wall formed by photopolymerization and a solution partition partitioned by the wall and capable of polymerization reaction. In addition, the kit according to an embodiment of the present disclosure may preferably further include a cross-linker. In addition, the kit according to an embodiment of the present disclosure may preferably further include a PCR enhancer. In addition, the kit according to an embodiment of the present disclosure may preferably further include PCR essential elements. In addition, the kit according to an embodiment of the present disclosure may further include a light-transmitting PCR container for containing the digital PCR solution.

The photopolymerizable monomer serves to form a gel polymer wall, preferably a hydrogel wall, having a predetermined pattern in the digital PCR solution when light is irradiated in a predetermined pattern in the light-transmitting PCR container containing the digital PCR solution. The digital PCR solution may be partitioned into a plurality of solution partitions by the gel polymer walls. The type of photopolymerizable monomer is not particularly limited as long as the photopolymerizable monomer forms a gel polymer that maintains a shape without flowing by photopolymerization, and may be selected from various known photopolymerizable monomers. For example, the photopolymerizable monomer may be at least one selected from the group consisting of acrylamide, methacrylamide, gelatin acrylate, gelatin methacrylate (GelMA), ethylene glycol dimethacrylate (EGDMA), ethylene glycol diacrylate (EGDA), acrylated hyaluronic acid, and methacrylated hyaluronic acid.

The photoinitiator absorbs light (e.g., ultraviolet or visible light) energy to generate free radicals and induce a polymerization reaction of the photopolymerizable monomers. The type of photoinitiator is not particularly limited as long as the photoinitiator may be easily dispersed in water or dissolved in water, and may be selected from various known photoinitiators. For example, the photoinitiator may be at least one selected from the group consisting of phenylbis(2,4,6-trimethylbenzoyl)phosphine oxide; 2-hydroxy-4′-(2-hydroxyethoxy)-2-methylpropiophenone; riboflavin; eosin-Y; 2,2′-azobis(2-methyl-N-(2-hydroxyethyl)propionamide; lithium phenyl-2,4,6-trimethylbenzoylphosphinate (LAP); diphenyl(2,4,6-trimethylbenzoyl)phosphine oxide [TPO]; and tetramethylethylenediamine (TEMED). In addition, commercial products of the photoinitiator include Irgacure series products such as Irgacure-2959, Irgacure-819, and Irgacure-500; Sinocure TPO; an azo photoinitiator VA-086, and the like. The photoinitiator is preferably provided in a powder form to maintain the activity.

The fluorescent probe serves to indicate the presence or amount of a target nucleic acid as the size of a fluorescent signal. The type of fluorescent probe is not particularly limited as long as the fluorescent probe is used for detection or quantification of nucleic acids, and may be selected from various known fluorescent emitting probes. For example, the fluorescent probe may be selected from a fluorescent dye that binds to double-stranded DNA or a fluorescent material-labeled oligonucleotide type hydrolysis probe. The fluorescent material-labeled oligonucleotide type hydrolysis probe is configured to include a reporter fluorescent dye at a 5′ end and a quencher dye at a 3′ end, and emits a fluorescent signal by binding to a specific DNA sequence. Commercial products of the fluorescent probe include SYBR Green I dye, EvaGreen dye, SYTO-82 dye, TaqMan probe, Amplifluor probe, etc.

The cross-linker cross-links polymers generated by the polymerization reaction of the photopolymerizable monomers to form a network structure, and serves to impart mechanical strength and chemical stability to the gel polymer wall. When a monomer having a bifunctional acrylic group or methacrylic group or a monomer in a partial polymer form is selected as the photopolymerizable monomer, the gel wall formed by the polymerization of the photopolymerizable monomers may have sufficient strength and chemical stability. Meanwhile, when acrylamide or methacrylamide is selected as the photopolymerizable monomer, the kit may preferably include a cross-linker to form a gel wall having appropriate strength and chemical stability. The kit according to an embodiment of the present disclosure preferably includes a combination of a photopolymerizable monomer selected from acrylamide or methacrylamide and a cross-linker selected from N,N′-methylenebisacrylamide, etc.

In the digital PCR solution, a photopolymerizable monomer or a photoinitiator may be present, which may decrease the amplification efficiency during the PCR. The PCR enhancer serves to increase the activity of DNA polymerase or stabilize DNA polymerase to alleviate the decrease in amplification efficiency. The PCR enhancer may be selected from various known additives to be added to increase the amplification efficiency or improve the specificity during the PCR. For example, the PCR enhancer may be at least one selected from the group consisting of nonionic surfactants, bovine serum albumin (BSA), skim milk, gelatin, casein, serum, and dimethyl sulfoxide (DMSO). The type of nonionic surfactant is not particularly limited as long as the nonionic surfactant increases the activity of DNA polymerase, and may be selected from, for example, polyoxyethylene (20) sorbitan monolaurate, polyoxyethylene (20) sorbitan monooleate, t-octylphenoxypolyethoxyethanol, etc. Commercial products of the nonionic surfactant include Tween 20, Tween 80, NP-40, Triton X-100, etc.

The PCR essential elements are a combination of components essentially required for amplification of a target nucleic acid, and consist of DNA polymerase, deoxynucleoside triphosphate (dNTP), a metal ion-providing compound, and a buffer solution. The type of metal ion-providing compound is not particularly limited as long as the metal ion-providing compound is a compound for providing a metal ion that acts as an essential cofactor of DNA polymerase in an aqueous solution, and may be selected from compounds capable of providing, for example, magnesium ions (Mg²⁺) or manganese ions (Mn²⁺). The metal ion-providing compound includes magnesium salts such as MgCl₂, manganese salts such as MnCl₂, etc. In addition, the PCR essential elements may further include reverse transcriptase to perform reverse transcription PCR when the target nucleic acid is RNA. The PCR essential elements may preferably be provided in the form of a PCR master mix.

The type of light-transmitting PCR container is not particularly limited as long as the light-transmitting PCR container has a predetermined space for containing the digital PCR solution and is made of a material capable of transmitting light such as ultraviolet rays, and for example, may be selected from a glass slide combined with an imaging spacer, a microfluidic chip, a cuvette, a small transparent plastic container, etc.

One aspect of the present disclosure relates to a method for detecting or quantifying nucleic acids based on photopolymerization.

The method for detecting or quantifying nucleic acids according to an embodiment of the present disclosure includes (a) preparing a light-transmitting PCR container containing a digital PCR solution; (b) irradiating light to the light-transmitting PCR container containing the digital PCR solution to induce photopolymerization so that the digital PCR solution is partitioned into a pattern in which solution partitions and gel walls are repeated, and generate a plurality of isolators; (c) applying heat to the light-transmitting PCR container to amplify nucleic acids present in the solution partitions for each isolator after partitioning the digital PCR solution; and (d) acquiring fluorescence data from the partitioned isolators using a fluorescence imaging technique, after amplifying the nucleic acids.

The digital PCR solution includes a target nucleic acid-containing sample, a primer, a photopolymerizable monomer, a photoinitiator, a fluorescent probe, a DNA polymerase, a deoxynucleoside triphosphate (dNTP), a metal ion-providing compound, and a buffer solution. In addition, the digital PCR solution may optionally further include at least one selected from the group consisting of preferably a cross-linker, a PCR enhancer, and a reverse transcriptase. The specific technical characteristics of the components constituting the digital PCR solution refer to those described above.

The target nucleic acid-containing sample constituting the digital PCR solution may be at least one selected from the group consisting of cells, tissues, blood, serum, urine, and saliva isolated from a specimen. In addition, the target nucleic acid-containing sample is preferably a nucleic acid extract extracted from cells, tissues, blood, serum, urine, or saliva isolated from the specimen. The nucleic acid extract may be prepared using various commercial nucleic acid extraction kits. In addition, the target nucleic acid includes all kinds of nucleic acids to be detected. In addition, the nucleic acid includes not only single-stranded molecules, but also double-stranded or triple-stranded nucleic acids, and may include not only DNA and RNA, but also nucleotide analogs.

In the digital PCR solution, the concentration of the photopolymerizable monomer or the combined concentration of the photopolymerizable monomer and the cross-linker is not particularly limited, but considering the mechanical strength, chemical stability, etc. of the gel wall, the concentration is preferably 1 to 30% (w/v), and more preferably 5 to 20% (w/v). In general, a polyacrylamide gel having a concentration of 5 to 10% (w/v) is used in DNA electrophoresis to check the quality of the PCR result. On the gel wall having a polyacrylamide concentration of 6% (w/v), the migration of a target nucleic acid of 500 bp or longer may be restricted, and on the gel wall having a polyacrylamide concentration of 10% (w/v) or higher, long nucleic acid molecules are separated into respective solution spaces, so that crosstalk of target nucleic acids may be prevented during the digital PCR process. However, the concentration of the photopolymerizable monomer or the combined concentration of the photopolymerizable monomer and the cross-linker in the digital PCR solution may be selected in various ranges considering the size of target nucleic acid, the type of photopolymerizable monomer, the thickness of gel wall, etc.

It is preferable that the inner surface of the light-transmitting PCR container is modified by silanization before the digital PCR solution is contained. The light-transmitting PCR container with the inner surface modified by silanization may be used to prevent nucleic acids from being adsorbed to the inner surface of the light-transmitting PCR container. The silanization is a method of imparting hydrophobicity to the surface by substituting a hydroxyl group present on the surface with inactive alkylsiloxy. The gel is covalently bonded to the inner surface of the container by the silanization, thereby preventing the gel partition from being deformed during the amplification process by a thermal cycle, and minimizing the diffusion of the amplified amplicon to the surroundings. The type of light-transmitting PCR container is not particularly limited as long as the light-transmitting PCR container has a predetermined space for containing the digital PCR solution and is made of a material capable of transmitting light such as ultraviolet rays, and for example, may be selected from a glass slide combined with an imaging spacer, a chip provided with microfluidic channels, a chip provided with microfluidic chambers, a cuvette, a small transparent plastic container, etc.

In step (b), the partitioning of the digital PCR solution may be performed by photolithography. Specifically, the partitioning of the digital PCR solution may be performed by a photolithography system consisting of a photomask with a predetermined pattern and an ultraviolet lamp or a light-sheet photolithography system. In step (b), when light is irradiated in a predetermined pattern onto the light-transmitting PCR container containing the digital PCR solution, a photopolymerization reaction occurs in a portion through which light is transmitted, and a gel polymer wall (e.g., hydrogel wall) having a width corresponding to a gap of the photomask or a thickness of the light sheet is formed. Considering the induction of a smooth photopolymerization reaction, the wavelength of the light or the light sheet used in step (b) is preferably 200 to 405 nm, and more preferably 350 to 400 nm. In addition, the thickness of the light sheet used in step (b) is not particularly limited, and is preferably 1 to 1000 μm, and more preferably 10 to 200 μm when considering the width of the gel wall.

In step (b), the digital PCR solution may be partitioned 1, 2, or 3-dimensionally within the light-transmitting PCR container. For example, when the microfluidic chip with microfluidic channels is used as the light-transmitting PCR container, the digital PCR solution may be partitioned into a 1-dimensional grid pattern in which solution partitions and gel walls surrounding or partitioning the solution partitions are repeated in one direction (e.g., X-axial direction). In addition, when the microfluidic chip with microfluidic chambers is used as the light-transmitting PCR container, the digital PCR solution may be partitioned into a 1-dimensional grid pattern in which solution partitions and gel walls surrounding the solution partitions are repeated in two directions (e.g., X-axial direction and Y-axial direction). In addition, when the cuvette is used as the light-transmitting PCR container, the digital PCR solution may be partitioned into a 1-dimensional grid pattern in which solution partitions and gel walls surrounding the solution partitions are repeated in three directions (e.g., X-axial direction, Y-axial direction, and Z-axial direction).

In the case of partitioning the digital PCR solution using the light-sheet photolithography system in step (b), each partition may also be generated one by one, or a plurality of partitions may also be simultaneously generated by applying interference lithography technology. When partitioning the digital PCR solution using the light-sheet photolithography system, the size and number of partitions may be easily changed by changing an optical system, such as slits, cylindrical lenses, etc. of the optical system, and by adjusting the moving distance of a stage.

Step (c) is preferably performed in a thermal cycler to apply heat to the light-transmitting PCR container with a PCR temperature profile. In addition, the process of applying heat to the light-transmitting PCR container in step (c) is preferably performed in the dark with blocked light to prevent the solution partitions of the non-photopolymerized digital PCR solution from being photopolymerized. In addition, in step (c), it is preferable to uniformly distribute the temperature of the plurality of isolators by applying heat in all directions, such as the bottom, sides, and top of the light-transmitting PCR container. In addition, step (c) may further include a process of irradiating the entire light-transmitting PCR container with light to induce the photopolymerization reaction and fix the amplified nucleic acids inside the isolators, after amplifying the nucleic acids.

The fluorescence imaging technique used to acquire fluorescence data in step (d) may be implemented by a fluorescence imaging system. The type of fluorescence imaging system is not particularly limited, and considering the ease and speed of fluorescence signal measurement, the fluorescence imaging system is preferably a confocal laser scanning microscope (CLSM) or a light-sheet fluorescence microscope (LSFM). The light-sheet fluorescence microscope (LSFM) may capture a fluorescence signal image of the 3-dimensionally partitioned digital PCR solution for each isolator layer and 3-dimensionally reconstruct the results thereof. For example, in step (b), when the digital PCR solution is 3-dimensionally partitioned in the light-transmitting PCR container by the light-sheet photolithography system, the acquiring of the fluorescence data in step (d) may be performed by irradiating a light sheet on each isolator layer consisting of a plurality of isolators to acquire fluorescence images 2-dimensionally partitioned for each isolator layer, and stacking the fluorescence images for each isolator layer to reconstruct the images 3-dimensionally.

The method for detecting or quantifying the nucleic acids according to an embodiment of the present disclosure may preferably further include (e) calculating the concentration of the specimen nucleic acids based on Poisson statistics from the acquired fluorescence data.

When using the method for detecting or quantifying the nucleic acids according to an embodiment of the present disclosure, the digital PCR solution may be partitioned into 10³ to 10⁸ isolators within the light-transmitting PCR container. When using the method for detecting or quantifying the nucleic acids according to an embodiment of the present disclosure, the sample loss is very small and the nonuniformity of the partitioned isolators hardly occurs during the process of partitioning the digital PCR solution.

One aspect of the present disclosure relates to a device for detecting or quantifying nucleic acids based on photopolymerization.

The device for detecting or quantifying nucleic acids according to an embodiment of the present disclosure includes the kit for preparing the digital PCR solution described above. In addition, the device for detecting or quantifying nucleic acids according to an embodiment of the present disclosure may optionally further include a photolithography system or a fluorescence imaging system. The photolithography system is preferably a light-sheet photolithography system. In addition, the fluorescence imaging system may be selected from a fluorescence microscope or a confocal laser scanning microscope (CLSM). In addition, the fluorescence microscope is preferably a light-sheet fluorescence microscope (LSFM).

Hereinafter, the present disclosure will be described in more detail with reference to Examples. However, the following Examples are only for clearly illustrating the technical features of the present disclosure, but do not limit the protection scope of the present disclosure.

1. Preparation of Polymerase Chain Reaction (PCR) Solution that May be Partitioned by Photopolymerization

In an Eppendorf LoBind tube, components listed in Table 1 below were added in deionized water to have a predetermined concentration and mixed to prepare a PCR solution.

TABLE 1

| Component | Final concentration |
|---|---|
| Acrylamide | 9.5% (w/v) |
| N,N'-Methylenebisacrylamide | 0.5% (w/v) |
| LAP (photoinitiator) | 0.01% (w/v) |
| Tween 20 | 4% (v/v) |
| Forward primer | 10 μM |
| Reverse primer | 10 μM |
| SYBR Green I | 1X |
| HiSense HotTaq PCR master mix (CellSafe Co., Ltd.) | 1X |
| λ gDNA fragment | variable |

LAP (photoinitiator): Lithium phenyl-2,4,6-trimethylbenzoylphosphinate
Tween 20: Commercial product name of Polyoxyethylene (20) sorbitan monolaurate
Forward primer: SEQ ID NO: 1
Reverse prime: SEQ ID NO: 2
HiSense HotTaq PCR master mix: Product in the form of master mix consisting of Taq DNA Polymerase, Taq antibody, Deoxynucleoside triphosphates (dNTPs), MgCl₂ and buffer solution
λ gDNA fragment: 364-bp partial sequence consisting of a base sequence represented by SEQ ID NO: 3 in gDNA (48,000 bp) of bacteriophage λ, which is designed so that in Example of nucleic acid detection and quantification using 1D dPCR to be described below, the final concentration of the λ gDNA fragment is 20.31 fM, and in Example of nucleic acid detection and quantification using 2D PCR, the final concentration of the λ gDNA fragment is 83 aM and 166 aM

### 2. Fabrication of Light-Sheet Photolithography System

A light-sheet photolithography (LSP) system was fabricated to variably form a plurality of spaces partitioned into a grid pattern as desired by irradiating a polymerase chain reaction (PCR) solution contained in a predetermined space with a light sheet at a predetermined interval and in a predetermined direction without using a photomask.

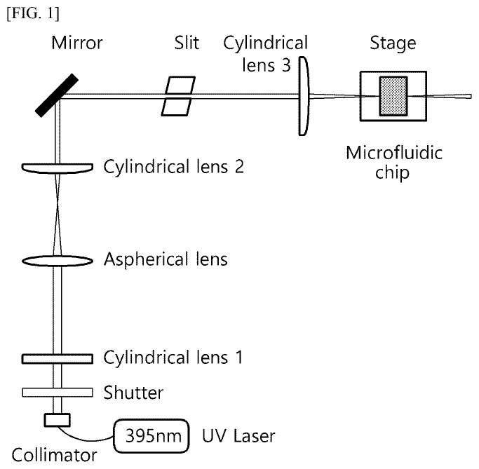

FIG. 1 schematically illustrates a configuration of a light-sheet photolithography system. The light-sheet photolithography system consists of 1) a UV optical fiber laser having a wavelength of 395 nm suitable for a water-soluble photoinitiator, 2) a collimator for expanding the optical fiber laser beam, 3) a shutter, 4) a cylindrical lens 1 (f=25 mm), 5) an aspherical lens (f=8 mm), 6) a cylindrical lens 2 (f=100 mm), 7) a mirror, 8) a slit, and 9) a cylindrical lens 3 (f=150 mm). The light-sheet photolithography system is disposed on a motorized stage to irradiate a light sheet having a wavelength of 395 nm and a width of 50 μm to a specific space at a constant interval while moving in a predetermined direction.

3. Detection and Quantification of Nucleic Acids Using Photopolymerization-Based 1D dPCR
(1) Preparation of PCR Solution that May be Partitioned by Photopolymerization

A PCR solution was prepared by setting the final concentration of the λ gDNA fragment to 20.31 fM in Table 1 above.

(2) Preparation of 1D dPCR Microfluidic Chip and Surface Modification of Microfluidic Channel

A 1D dPCR microfluidic chip having linear microfluidic channels with a length of 45.6 mm, a width of 50 μm, and a depth of 20 μm was prepared to partition a PCR solution 1-dimensionally. In addition, a surface-modification solution was prepared by mixing 3-(trimethoxysilyl)propyl methacrylate, acetic acid, methanol, and deionized water in a volume ratio of 2:2:3:3. First, the microfluidic channels of the 1D dPCR microfluidic chip were washed sequentially with deionized water, a TN sodium hydroxide solution, and deionized water, and then dried. Then, the surface-modification solution was filled into the microfluidic channels of the 1D dPCR microfluidic chip, and reacted for 30 minutes. Thereafter, the surface-modification solution was removed from the microfluidic channels, and washed with methanol for 10 minutes, and dried to prepare a 1D dPCR microfluidic chip in which the surface of the microfluidic channel was modified by alkoxysilane.

### (3) 1-Dimensional Partitioning of PCR Solution Using Photopolymerization

The PCR solution was filled into the surface-modified microfluidic channel of the 1D dPCR microfluidic chip, and a polydimethylsiloxane (PDMS) solution was filled into the inlet and outlet wells at both ends of the microfluidic channel, and then each well was sealed with a PCR tape (PET/acrylic adhesive film; AB-0558, Thermo Fisher Scientific). In addition, a photomask was prepared to have a grid pattern in which 100 μm wide chrome stripes and 37.5 μm wide gaps were alternately repeated. Thereafter, the microfluidic chip was aligned and placed on the photomask and irradiated with UV light from the bottom using a 365 nm wavelength UV lamp (13 mW/cm², M365LP1-C1, Thorlabs) for about 40 minutes. In a space corresponding to the gap of the photomask among the microfluidic channel spaces, photopolymerization was performed due to UV irradiation, and the PCR solution was gelled. When the UV irradiation was completed, the PCR solution filled in the microfluidic channel was partitioned into a 1-dimensional grid pattern having repeated solution partitions with a width of 100 μm and gel walls with a width of 37.5 μm, and about 331 isolators were generated in the microfluidic channel.

Meanwhile, the PCR solution filled in the surface-modified microfluidic channel of the microfluidic chip may be partitioned into a plurality of isolators by a light-sheet photolithography system instead of a photolithography system consisting of a photomask and an ultraviolet lamp. A 1-dimensional partitioning method of the PCR solution using the light-sheet photolithography system is as follows.

After filling the PCR solution in the surface-modified microfluidic channel of the 1D dPCR microfluidic chip, the microfluidic chip is fixed to an XZ plane of a motorized stage.

While the motorized stage is moved in an X-axial direction at a constant interval (e.g., 100 μm), a light sheet that is perpendicular to the XZ plane and horizontal to a YZ plane is irradiated for about 20 minutes to repeatedly form 100 μm wide solution partitions and 50 m wide gel walls in a longitudinal direction of the microfluidic channel. Finally, the PCR solution filled in the microfluidic channel is partitioned into a 1-dimensional grid pattern in which the 100 μm wide solution partitions and 50 μm wide gel walls are repeated, and a plurality of isolators are generated in the microfluidic channel.

### (4) Amplification of Nucleic Acids Using Thermal Cycler

A PCR solution was partitioned into a 1-dimensional grid pattern by using a photolithography system consisting of a photomask and an ultraviolet lamp, and then a 1D dPCR microfluidic chip was mounted on a thermal cycler and heated with a PCR temperature profile as shown in Table 2 below to amplify nucleic acids present in the solution partition for each isolator.

TABLE 2

| Step | Temperature | Time | Cycles |
|---|---|---|---|
| Pre-denaturation | 95° C. | 5 min | 1 cycle |
| Denaturation | 95° C. | 30 sec | 31 cycles |
| Annealing | 56.1° C. | 30 sec | |
| Extension | 72° C. | 30 sec | |
| Final extension | 72° C. | 5 min | 1 cycle |

### (5) Extraction of Fluorescence Signals and Quantification of Nucleic Acids

After completing the amplification of nucleic acids using a thermal cycler, a PCR tape was removed from the microfluidic chip, and the microfluidic chip was inserted into a custom 3D-printed chip insert compatible with an inverted fluorescence microscope stage (IX73, Olympus). Thereafter, fluorescence images were acquired by scanning the entire microfluidic channels of the microfluidic chip using the inverted fluorescence microscope fitted with a motorized XY stage (MS-2000, ASI), an sCMOS camera (Zyla, Andor), and Metamorph software (Molecular Devices). Thereafter, the fluorescence signal values were extracted from the acquired fluorescence images using Origin Pro software (OriginLab Corp.), and the nucleic acids were quantified using the Poisson statistics equation. The concentration of the target DNA may be calculated by the following Equation 1.

$${C}_{DNA} = \frac{- ln ( 1 - P )}{{v}_{d}} \qquad [ Equation 1 ]$$

In Equation 1, C_{DNA} is a measured concentration of target DNA, P is the fraction of the number of isolators showing a positive fluorescence signal compared to the total number of isolators, and v_{d} is the volume of each isolate. The volume occupied by the gel wall in the isolator volume is excluded from the calculation.

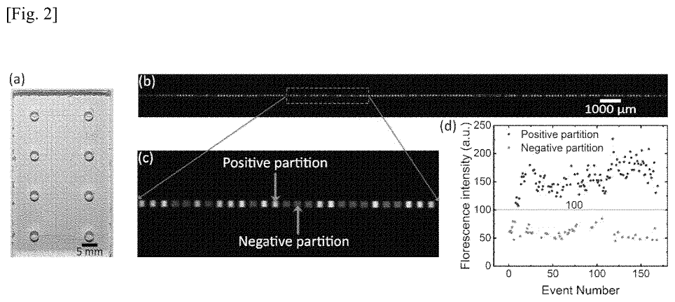

As a result of quantification using Poisson statistics, the experimented λ-gDNA template concentration was calculated to be 2018 copies/μl. FIG. 2 is a result acquired by performing a process for detecting and quantifying nucleic acids using 1D dPCR in an embodiment of the present disclosure. FIG. 2A is an image of a 1D dPCR microfluidic chip made of glass, FIG. 2B is a fluorescence image acquired by scanning a microfluidic channel after completing amplification of nucleic acids using a thermal cycler, FIG. 2C is an enlarged portion of the fluorescence image of FIG. 2B, and FIG. 2D illustrates a distribution of fluorescence signal intensities of each isolator partitioned in a microfluidic channel when the amplification of nucleic acids using the thermal cycler is completed.

4. Detection and Quantification of Nucleic Acids Using Photopolymerization-Based 2D dPCR
(1) Preparation of Polymerase Chain Reaction (PCR) Solution that May be Partitioned by Photopolymerization

In Table 1 above, the final concentrations of the λ gDNA fragment were set to 83 aM and 166 aM to prepare two PCR solutions with different concentrations.

(2) Preparation of 2D dPCR Microfluidic Chip and Surface Modification of Microfluidic Chamber

A 2D dPCR microfluidic chip having microfluidic chambers with a width of 7500 μm, a length of 7500 μm, and a depth of 100 μm was prepared to partition a PCR solution 2-dimensionally. In addition, the 2D dPCR microfluidic chip in which the inner surface of the microfluidic chamber was modified with alkoxysilane was prepared using the same method as the method for modifying the surface of the microfluidic channel of the 1D dPCR microfluidic chip.

### (3) 2-Dimensional Partitioning of PCR Solution Using Photopolymerization

A PCR solution was filled into the surface-modified microfluidic chamber of the 2D dPCR microfluidic chip, and a polydimethylsiloxane (PDMS) solution was filled into the inlet and outlet wells at both ends of the microfluidic chamber, and then each well was sealed with a PCR tape (PET/acrylic adhesive film; AB-0558, Thermo Fisher Scientific). In addition, a photomask was prepared to have a grid pattern in which 100 μm×100 μm chrome squares and 37.5 μm wide gaps surrounding the chrome squares were alternately repeated. Thereafter, the microfluidic chip was aligned and placed on the photomask and irradiated with UV light from the bottom using a 365 nm wavelength UV lamp (13 mW/cm², M365LP1-C1, Thorlabs) for about 40 minutes. In a space corresponding to the gap of the photomask among the microfluidic chamber spaces, photopolymerization was performed due to UV irradiation, and the PCR solution was gelled. When the UV irradiation was completed, the PCR solution filled in the microfluidic chamber was partitioned into a 2-dimensional grid pattern in which 100 μm×100 μm square solution partitions and 37.5 μm wide gel walls surrounding the solution partitions were repeated, and about 2916 isolators were generated in the microfluidic channel.

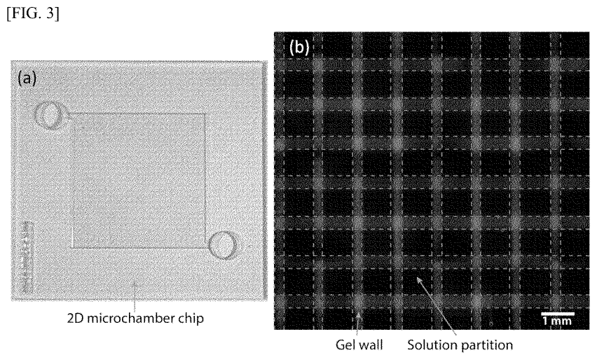

FIG. 3 is a schematic diagram of a microfluidic chip used when performing a process for detecting and quantifying nucleic acids using 2D dPCR in an embodiment of the present disclosure, and isolators in a 2-dimensional grid pattern formed in a microfluidic chamber of the microfluidic chip. FIG. 3A is an image of a glass 2D dPCR microfluidic chip used when performing a process of detecting and quantifying nucleic acids using 2D dPCR in an embodiment of the present disclosure, and FIG. 3B is a schematic diagram of isolators in a 2-dimensional grid pattern formed when a PCR solution filled in a microfluidic chamber of a 2D dPCR microfluidic chip is partitioned using a photolithography system consisting of a photomask and an ultraviolet lamp in an embodiment of the present disclosure.

Meanwhile, the PCR solution filled in the surface-modified microfluidic chamber of the microfluidic chip may be partitioned into a plurality of isolators even by a light-sheet photolithography system instead of the photolithography system consisting of the photomask and the ultraviolet lamp. A 2-dimensional partitioning method of the PCR solution using the light-sheet photolithography system is as follows.

After filling the PCR solution in the surface-modified microfluidic chamber of the 2D dPCR microfluidic chip, the microfluidic chip is fixed to an XZ plane of a motorized stage.

While the motorized stage is moved in an X-axial direction at a constant interval (e.g., 100 μm), a light sheet that is perpendicular to the XZ plane and horizontal to a YZ plane is irradiated for about 20 minutes to repeatedly form 100 μm wide solution partitions and 50 m wide gel walls in a horizontal direction of the microfluidic chamber.

After rotating the microfluidic chip by 90°, the microfluidic chip is fixed to the XZ plane of the motorized stage again. While the motorized stage is moved in an X-axial direction at a constant interval (e.g., 100 μm), a light sheet that is perpendicular to the XZ plane and horizontal to a YZ plane is irradiated for about 20 minutes to repeatedly form 100 μm wide solution partitions and 50 μm wide gel walls in a longitudinal direction of the microfluidic chamber. Finally, the PCR solution filled in the microfluidic chamber is partitioned into a 2-dimensional grid pattern in which 100 μm×100 μm square solution partitions and 50 μm wide gel walls surrounding the solution partitions are repeated, and a plurality of isolators are generated in the microfluidic chamber.

### (4) Amplification of Nucleic Acids Using Thermal Cycler

A PCR solution was partitioned into a 2-dimensional grid pattern using a photolithography system consisting of a photomask and an ultraviolet lamp, and the 2D dPCR microfluidic chip was mounted on a thermal cycler and heated with a PCR temperature profile shown in Table 2 above to amplify the nucleic acids present in the solution partition for each isolator.

### (5) Extraction of Fluorescence Signals and Quantification of Nucleic Acids

The entire microfluidic chamber of the 2D dPCR microfluidic chip was scanned and fluorescence images were acquired using the same method as the method performed in the process of detecting and quantifying the nucleic acids using 1D dPCR. Thereafter, fluorescence signal values were extracted from the fluorescence images, and the nucleic acids were quantified using Equation 1 applied with the Poisson statistics equation.

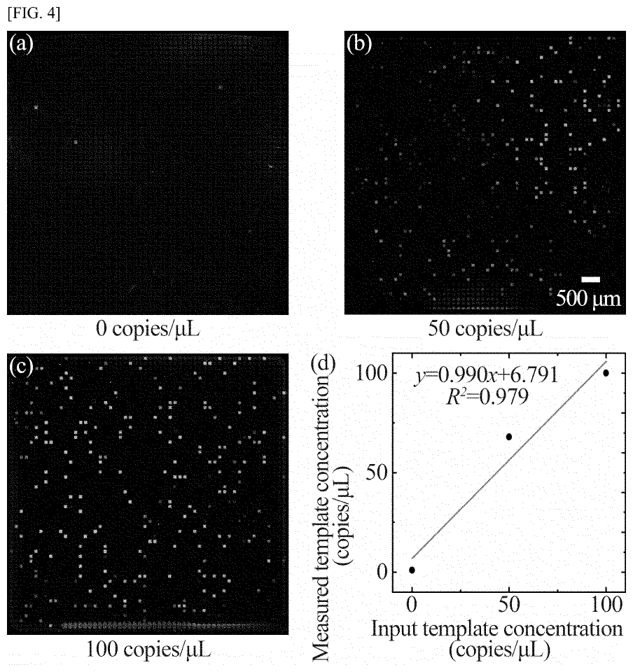

FIG. 4 is a result acquired by performing a process for detecting and quantifying nucleic acids using 2D dPCR in an embodiment of the present disclosure. FIG. 4A is a fluorescence image acquired by scanning a microfluidic chamber after partitioning a PCR solution without λ-gDNA into a 2-dimensional grid shape using photopolymerization and completing amplification of nucleic acids using a thermal cycler; FIG. 4B is a fluorescence image acquired by scanning a microfluidic chamber after partitioning a PCR solution having a λ-gDNA concentration of 83 aM into a 2-dimensional grid shape using photopolymerization and completing amplification of nucleic acids using a thermal cycler; FIG. 4C is a fluorescence image acquired by scanning a microfluidic chamber after partitioning a PCR solution having a λ-gDNA concentration of 166 aM into a 2-dimensional grid shape using photopolymerization and completing amplification of nucleic acids using a thermal cycler; and FIG. 4D is a graph showing a linear relationship between a λ-gDNA concentration of the PCR solution and a λ-gDNA concentration measured in the process of detecting and quantifying nucleic acids using 2D dPCR. As shown in FIG. 4D, when the process of detecting and quantifying nucleic acids was performed using 2D dPCR, the R² value was 0.979, which was almost close to 1, and this result means that the accuracy of the method for detecting or quantifying nucleic acids based on photopolymerization technology proposed in the present disclosure is very high.

5. Detection and Quantification of Nucleic Acids Using Photopolymerization-Based 3D dPCR

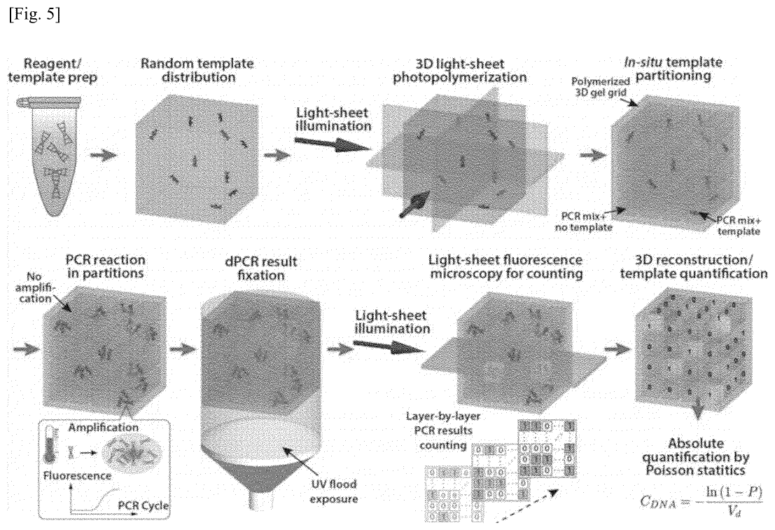

In the case of using the method for detecting and quantifying nucleic acids based on photopolymerization technology proposed in the present disclosure, a polymerase chain reaction (PCR) solution may be partitioned into a 3-dimensional grid pattern to generate thousands to millions of isolators. FIG. 5 is a schematic diagram illustrating a method for detecting and quantifying nucleic acids using a photopolymerization-based 3D dPCR proposed in the present disclosure step by step. As shown in FIG. 5, a method for detecting and quantifying nucleic acids using a photopolymerization-based 3D dPCR largely consists of the steps of preparing a PCR solution that may be partitioned by photopolymerization and selecting a container containing the PCR solution; 3-dimensionally partitioning the PCR solution using a light-sheet photolithography system; amplifying nucleic acids through PCR thermal cycling; and measuring fluorescence signals and quantifying nucleic acids using light-sheet fluorescence microscopy (LSFM).

(1) Preparation of PCR Solution that May be Partitioned by Photopolymerization and Selection of Container Containing PCR Solution

The PCR solution that may be partitioned by photopolymerization essentially includes a target nucleic acid-containing sample, a primer, a fluorescent probe, a photopolymerizable monomer capable of forming a gel (particularly, hydrogel) polymer by photopolymerization, a photoinitiator, and preferably, further includes a cross-linker, a PCR enhancer, etc., in addition to a conventional PCR master mix consisting of DNA polymerase (Taq DNA Polymerase), deoxynucleoside triphosphates (dNTPs), MgCl₂, and a buffer solution. In the method for detecting and quantifying nucleic acids using photopolymerization-based 3D dPCR, the container containing the PCR solution needs to have a high light transmittance and an internal space that may be partitioned 3-dimensionally, such as a cuvette and the like used in spectroscopic measurements.

### (2) 3-Dimensional Partitioning of PCR Solution Using Light-Sheet Photolithography System

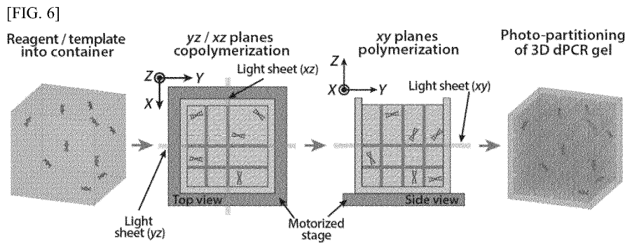

FIG. 6 illustrates a process for generating a plurality of isolators by 3-dimensionally partitioning a polymerase chain reaction (PCR) solution contained in a predetermined container using a light-sheet photolithography system. As shown in FIG. 6, three light-sheet photolithography units are used to 3-dimensionally partition a PCR solution contained in a cuvette. The cuvette containing the PCR solution is fixed to an XZ plane, and irradiated with a light sheet horizontal to a YZ plane and a light sheet horizontal to the XZ plane at preset intervals to be first partitioned into a 2-dimensional grid pattern in which square solution partitions and gel walls surrounding the solution partitions are repeated, and then irradiated with a light sheet horizontal to the XY plane at preset intervals to be partitioned into a 3-dimensional grid pattern in which solution partitions in a regular hexahedron shape (for example, a cube having all width, length, and height of 100 μm) and gel walls (for example, gel walls having a width of 50 μm) surrounding the solution partitions are repeated, and thus a plurality of isolators are generated in the cuvette. When the PCR solution is partitioned 3-dimensionally using the light-sheet photolithography system, 10³ to 10⁶ high-density isolators may be generated.

### (3) Amplification of Nucleic Acids Using PCR Thermal Cycler

After partitioning the PCR solution into the 3-dimensional grid pattern, the 3D dPCR container (e.g., cuvette) is mounted in a thermal cycler and heated with a PCR temperature profile to amplify the nucleic acids present in the solution partition for each isolator.

### (4) Measurement of Fluorescence Signals and Quantification of Nucleic Acids Using Light-Sheet Fluorescence Microscopy (LSFM)

When detecting and quantifying nucleic acids using photopolymerization-based 3D dPCR, the isolators have a form in which spaces partitioned into the 2-dimensional grid pattern are vertically stacked in a 3-dimensional space, and thus a method for effectively counting positive and negative fluorescence signals within the 3-dimensional grid pattern is required. The detection of target DNA for each isolator in the PCR solution partitioned into the 1-dimensional or 2-dimensional grid pattern may be performed by acquiring fluorescence images using a standard fluorescence microscope and using commercial image analysis software. The acquiring of fluorescence data for each isolator in the PCR solution partitioned into the 3-dimensional grid pattern may be performed using a confocal laser scanning microscope (CLSM). However, the CLSM has a large size, a slow speed, and a typical vertical resolution of about 1 μm or less, and thus has unnecessarily excessive price and performance for capturing isolators consisting of relatively thick layers (tens to hundreds of m) in 3D dPCR. Therefore, a custom light-sheet fluorescence microscopy (LSFM) system is required to effectively acquire 2-dimensional image stacks of hundreds to thousands of partitioned isolator layers from the isolators partitioned into the 3-dimensional grid pattern and to reconstruct the results 3-dimensionally.

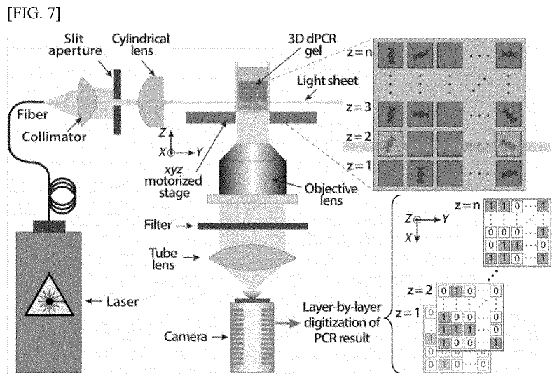

FIG. 7 illustrates a process for acquiring fluorescence data from isolators partitioned into a 3-dimensional grid pattern using a light-sheet fluorescence microscopy (LSFM) system. As shown in FIG. 7, the LSFM system has a structure similar to that of a light-sheet photolithography (LSP) system and may be constructed on an inverted microscope having a motorized stage. An optical system of the LSFM system is similar to that of the LSP system, but differs in that a laser having a wavelength of 488 nm, instead of ultraviolet light having a wavelength of 395 nm, is used to excite a fluorescent probe. The beam width of the laser irradiated in the LSFM system is preferably about 100 μm considering the thickness of the isolator in each layer. The process for acquiring the fluorescence data from the isolators partitioned into the 3-dimensional grid pattern using the LSFM system is as follows. First, when the light sheet is irradiated to a first layer (z=1) in the LSFM system, the fluorescent probe bound to DNA is excited to emit fluorescence only in positive isolations where the target DNA exists. The fluorescence image for each partition of each layer is captured by an objective lens and a high-sensitivity digital camera positioned perpendicular to the light sheet. Then, when the position of the motorized stage is lowered and the light sheet is irradiated, an image of a second isolator layer is captured. The process is repeated until the last isolator layer (z=n) is captured, and the images of the isolators are reconstructed 3-dimensionally using this continuous image sequence. The optical system, the motorized stage, and the image acquisition are performed under a computer control, and the dPCR results are counted based on the intensity of the fluorescence signal. Thereafter, the isolators having a fluorescence signal higher than a threshold value may be determined as a positive result, and the isolators having a fluorescence signal lower than the threshold value may be determined as a negative result. Finally, the concentration of target DNA, C_{DNA}, is calculated using the above-described equation 1 by applying Poisson statistics.

As described above, the present disclosure has been described through Examples above, but the present disclosure is not necessarily limited thereto, and various modifications may be made without departing from the scope and spirit of the present disclosure. Therefore, the scope of the present disclosure should be construed to include all embodiments falling within the scope of claims appended hereto.

1: A kit for preparing a digital polymerase chain reaction (PCR) solution comprising a photopolymerizable monomer, a photoinitiator, and a fluorescent probe,
wherein the photopolymerizable monomer forms a gel polymer by photopolymerization,
the digital PCR solution is partitioned into a plurality of isolators by photopolymerization, and
the isolator consists of a gel wall formed by photopolymerization and a solution partition partitioned by the wall and capable of polymerization reaction.

2: The kit of claim 1, wherein the photopolymerizable monomer is at least one selected from the group consisting of acrylamide, methacrylamide, gelatin acrylate, gelatin methacrylate (GelMA), ethylene glycol dimethacrylate (EGDMA), ethylene glycol diacrylate (EGDA), acrylated hyaluronic acid, and methacrylated hyaluronic acid, and
the photoinitiator is at least one selected from the group consisting of phenylbis(2,4,6-trimethylbenzoyl)phosphine oxide; 2-hydroxy-4′-(2-hydroxyethoxy)-2-methylpropiophenone; riboflavin; eosin-Y; 2,2′-azobis(2-methyl-N-(2-hydroxyethyl)propionamide; lithium phenyl-2,4,6-trimethylbenzoylphosphinate (LAP); diphenyl(2,4,6-trimethylbenzoyl)phosphine oxide [TPO]; and tetramethylethylenediamine (TEMED).

3: The kit of claim 1, wherein the fluorescent probe is selected from a fluorescent dye that binds to double-stranded DNA or a fluorescent material-labeled oligonucleotide type hydrolysis probe.

4: The kit of claim 1, further comprising:
a cross-linker or a PCR enhancer.

5: The kit of claim 4, wherein the PCR enhancer is at least one selected from the group consisting of nonionic surfactants, bovine serum albumin (BSA), skim milk, gelatin,
casein, serum, and dimethyl sulfoxide (DMSO).

6: The kit of claim 1, further comprising:
PCR essential elements consisting of a DNA polymerase, a deoxynucleoside triphosphate (dNTP), a metal ion-providing compound, and a buffer solution.

7: A method for detecting or quantifying nucleic acids based on photopolymerization using the kit of claim 1, the method comprising:
(a) preparing a light-transmitting PCR container containing a digital PCR solution;
(b) irradiating light to the light-transmitting PCR container containing the digital PCR solution to induce photopolymerization so that the digital PCR solution is partitioned into a pattern in which solution partitions and gel walls are repeated, and generate a plurality of isolators;
(c) applying heat to the light-transmitting PCR container to amplify nucleic acids present in the solution partitions for each isolator after partitioning the digital PCR solution; and
(d) acquiring fluorescence data from the partitioned isolators using a fluorescence imaging technique, after amplifying the nucleic acids,
wherein the digital PCR solution includes a target nucleic acid-containing sample,
a primer, a photopolymerizable monomer, a photoinitiator, a fluorescent probe, a DNA polymerase, a deoxynucleoside triphosphate (dNTP), a metal ion-providing compound, and a buffer solution.

8: The method of claim 7, wherein the digital PCR solution further comprises at least one selected from the group consisting of a cross-linker, a PCR enhancer, and a reverse transcriptase.

9: The method of claim 7, wherein the inner surface of the light-transmitting PCR container is modified by silanization before the digital PCR solution is contained.

10: The method of claim 7, wherein the light-transmitting PCR container is selected from a glass slide combined with an imaging spacer, a chip provided with microfluidic channels,
a chip provided with microfluidic chambers, a cuvette, or a transparent plastic container.

11: The method of claim 7, wherein in step (b), the partitioning of the digital PCR solution is performed by a photolithography system consisting of a photomask and an ultraviolet lamp or a light-sheet photolithography system.

12: The method of claim 7, wherein step (c) further comprises a process of irradiating light the entire light-transmitting PCR container to induce a photopolymerization reaction by and fix the amplified nucleic acids inside the isolator, after amplifying the nucleic acids.

13: The method of claim 7, wherein a fluorescence imaging system for implementing the fluorescence imaging technique is a confocal laser scanning microscope (CLSM) or a light-sheet fluorescence microscope (LSFM).

14: The method of claim 7, wherein in step (b), when the digital PCR solution is 3-dimensionally partitioned in the light-transmitting PCR container by the light-sheet photolithography system, the acquiring of the fluorescence data in step (d) is performed by irradiating a light sheet on each isolator layer consisting of a plurality of isolators to acquire fluorescence images 2-dimensionally partitioned for each isolator layer, and stacking the fluorescence images for each isolator layer to reconstruct the images 3-dimensionally.

15: A device for detecting or quantifying nucleic acids, comprising the kit for preparing the digital PCR solution of claim 1.
